SEQUENCE LISTING (1 sequence)

SEQ ID NO: 1 - DNA, 57 bp, Artificial Sequence
  misc_feature: Biotin label attached
tccggcgcgccgttttcccagtcacgatttttttttttttttttttttttttttttt

(19) United States
(12) Patent Application Publication
(10) Pub. No.: US 2002/0146693 A1
(43) Pub. Date: Oct. 10, 2002

(54) Isolation and identification of secreted proteins

(76) Inventor: Gregory M. Landes, Livermore, CA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/773,861

(22) Filed: Feb. 1, 2001

Related U.S. Application Data
(60) Provisional application No. 60/180,582, filed on Feb. 4, 2000.

(51) Int. Cl. C12Q 1/68; C07H 21/04; A61K 48/00

(52) U.S. Cl. 435/6; 514/44; 536/23.2; 536/24.3; 435/320.1

(57) ABSTRACT

The present invention provides a method for isolating, identifying and cataloging partial messenger RNAs (mRNAs) or gene tags correlating to secreted and non-secreted proteins in a cell or tissue sample. The method requires obtaining a polynucleotide from a cellular homogenate, wherein the polynucleotide encodes the polypeptide and determining the sequence of the polynucleotide and its expression level. In essence, the method consists of two phases: (1) enrichment/purification of membrane-bound mRNA, and (2) sequence analysis. Neither of these processes, if performed independently of each other, would enable specific and rapid identification and enumeration of transcripts that encode secreted proteins and integral membrane bound proteins. This invention also provides computer-related systems and methods. More specifically, the invention provides a system and method for automatically generating a data base of gene tags corresponding to secreted and non-secreted proteins from cell or tissue samples and using the data base for filtering the tag counts from the samples into meaningful candidates for further testing and analysis.

### TECHNICAL FIELD

This invention is in the fields of molecular biology, cell biology and immunology.

### BACKGROUND OF THE INVENTION

The imminent acquisition of the sequence of the entire human genome will provide a wealth of information on gene and genome structure and organization. In order to use this vast wealth of genetic information in the prediction and treatment of human disease, the next step is to develop methods for the analysis of the data. In particular, methods are required which will allow one to distinguish global patterns of differential gene expression between different cells, or between different pathological stages of the same cell. Methods of this type are often denoted functional genomics.

It is well known that many, but not all genes present in a cell are expressed at any given time. Fundamental questions of biology require knowledge of which genes are transcribed and the relative abundance of transcripts in different cells. Typically, when and to what degree a given gene is expressed has been analyzed one gene at a time.

Thus, information regarding the identity of all expressed genes in a cell and the level of expression of these genes would facilitate the study of many cellular processes such as activation, differentiation, aging, viral transformation, morphogenesis, and mitosis. A comparison of the expressed genes of a particular cell or the same cell from various individuals or species, under the same or different environmental stimuli, provides valuable insight into the functional capabilities of the cell.

### DISCLOSURE OF THE INVENTION

The identification of cell surface markers and secreted proteins has significant value for the development of new therapeutic and diagnostic products. Current methods, however, even those proclaimed to be high-throughput, are relatively slow and are not comprehensive. Combining a methodology that yields mRNA preparations highly enriched for transcripts encoding signal peptide-containing proteins with a means of rapid identification and quantification of expressed sequences provides a high-throughput means of comprehensively identifying genes that encode integral membrane proteins and secreted proteins as well as quantifying their expression level. The present invention broadly provides a method for isolating, identifying and cataloging partial messenger RNAs (mRNAs) or gene tags correlating to secreted and non-secreted proteins in a cell or tissue sample. The method requires obtaining a polynucleotide from a cellular homogenate, wherein the polynucleotide encodes the polypeptide and determining the sequence of the polynucleotide and its expression level. In essence, the method consists of two phases: (1) enrichment/purification of membrane-bound mRNA, and (2) sequence analysis. Neither of these processes, if performed independently of each other, would enable specific and rapid identification and enumeration of transcripts that encode secreted proteins and integral membrane bound proteins.

This invention also provides computer-related systems and methods. More specifically, the invention provides a system and method for automatically generating a data base of gene tags corresponding to secreted and non-secreted proteins from cell or tissue samples and using the data base for filtering the tag counts from the samples into meaningful candidates for further testing and analysis.

### BRIEF DESCRIPTION OF THE FIGURES

FIG. 1 is a flowchart depicting one method to identify mRNA associated with the endoplasmic reticulum.

FIG. 2 is a flowchart depicting identification and quantification of transcripts that encode secreted and integral membrane proteins.

### MODES FOR CARRYING OUT THE INVENTION

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual,” second edition (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds. 1987); “Current Protocols in Molecular Biology” (F. M. Ausubel et al., eds., 1987, and periodic updates); “PCR: The Polymerase Chain Reaction,” (Mullis et al., eds., 1994); “Current Protocols in Immunology” (J. E. Coligan et al., eds., 1991).

Definitions

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein a second polynucleotide “corresponds to” another (a first) polynucleotide if it is related to the first polynucleotide by any of the following relationships:

1) The second polynucleotide comprises the first polynucleotide and the second polynucleotide encodes a gene product.

2) The second polynucleotide is 5′ or 3′ to the first polynucleotide in cDNA, RNA, genomic DNA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide, for example a promoter sequence. The first and second polynucleotide may be fragment of a gene coding for a gene product. The second polynucleotide may be an exon of the gene while the first polynucleotide may be an intron of the gene.

3) The second polynucleotide is the complement of the first polynucleotide.

The “genotype” of a cell refers to the genetic makeup of the cell and/or its gene expression profile. Modulation of the genotype of a cell can be achieved by introducing additional DNA or RNA either as episomes or as an integral part of the chromosomal DNA of the recipient cell. Altering the expression level, e.g. mRNA abundance, of a particular gene using agents that regulate gene expression, can also modulate the genotype.

A “database” denotes a set of stored data that represent a collection of sequences including nucleotide and peptide sequences, which in turn represent a collection of biological reference materials.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

“Oligonucleotide” refers to polynucleotides of between about 5 and about 100 nucleotides of single- or double-stranded DNA. Oligonucleotides are also known as oligomers or oligos and may be isolated from genes, or chemically synthesized by methods known in the art.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein. Throughout this specification, numbering of amino acids in a peptide or polypeptide is from amino terminus to carboxy terminus.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. (see, e.g., WO 95/27071). Ads are easy to grow and do not require integration into the host cell genome. Recombinant Ad-derived vectors, particularly those that reduce the potential for recombination and generation of wild-type virus, have also been constructed. See, WO 95/00655; and WO 95/11984. Wild-type AAV has high infectivity and specificity integrating into the host cell's genome. See, Hermonat and Muzyczka (1984) Proc. Natl. Acad. Sci. USA 81:6466-6470; and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

“PCR primers” refer to primers used in “polymerase chain reaction” or “PCR,” a method for amplifying a DNA base sequence using a heat-stable polymerase such as Taq polymerase, and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce exponential and highly specific amplification of the desired sequence. See, e.g., PCR 2: A PRACTICAL APPROACH, supra. PCR also can be used to detect the existence of the defined sequence in a DNA sample.

“Host cell” or “recipient cell” is intended to include any individual cell or cell culture which can be or have been recipients for vectors or the incorporation of exogenous nucleic acid molecules, polynucleotides and/or peptides (or polypeptides). It also is intended to include progeny of a single cell, and the progeny may not necessarily be completely identical (in morphology or in genomic or total DNA complement) to the original parent cell due to natural, accidental, or deliberate mutation. The cells may be procaryotic or eucaryotic, and include but are not limited to bacterial cells, yeast cells, animal cells, and mammalian cells, e.g., murine, rat, simian or human.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

As used herein, the terms “restriction endonucleases” and “restriction enzymes” refer to bacterial enzymes that bind to a specific double-stranded DNA sequence termed a recognition site or recognition nucleotide sequence, and cut double-stranded DNA at or near the specific recognition site. “Type IIS” restriction endonucleases are those which cleave at a defined distance (up to 20 bases away) from their recognition sites. Endonucleases will be known to those of skill in the art. See, for example, CURRENT PROTOCOLS IN MOLECULAR BIOLOGY, Vol. 2, 1995, Ausubel et al. eds., Greene Publish. Assoc. & Wiley Interscience, Unit 3.1.15; New England Biolabs Catalog (1995).

A “sequence tag” or “SAGE tag” is a short sequence, generally under about 20 nucleotides, that occurs in a certain position in messenger RNA. The tag can be used to identify the corresponding transcript and gene from which it was transcribed. A “ditag” is a dimer of two sequence tags.

An “isolated” population of cells is “substantially free” of cells and materials with which it is associated in nature. By “substantially free” or “substantially pure” means at least 50% of the population are the desired cell type, preferably at least 70%, more preferably at least 80%, and even more preferably at least 90%.

A “composition” is intended to mean a combination of active agent and another compound or composition, inert (for example, a detectable agent, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARMACEUTICAL SCIENCES, 15TH ED. (Mack Publ. Co., Easton (1975)).

An “effective amount” is an amount sufficient to effect beneficial or desired results. An effective amount can be administered in one or more administrations, applications or dosages.

The present invention is directed to a method for identifying a polynucleotide sequence and its expression level, wherein the polynucleotide corresponds to an endoplasmic reticulum-associated polypeptide. The method requires obtaining a polynucleotide from a cellular homogenate, wherein the polynucleotide encodes the polypeptide and determining the sequence of the polynucleotide and its expression level.

In one aspect, the polynucleotide is isolated from a cell or tissue homogenate by isopycnic centrifugation and separation of the microsomal fraction. Methods of isolating such polynucleotides are known in the art, and described, for example, in Hedrick et al. (1984) Nature 308:149153; Hirama et al. (1986) Anal. Biochem. 155:385-390; Winemiller et al. (1989) Nucl. Acids Res. 17(12):4896; Hegde et al. (1997) Cell 91:575-582; and Mechler (1987) Meth. Enzymol. 152:241.

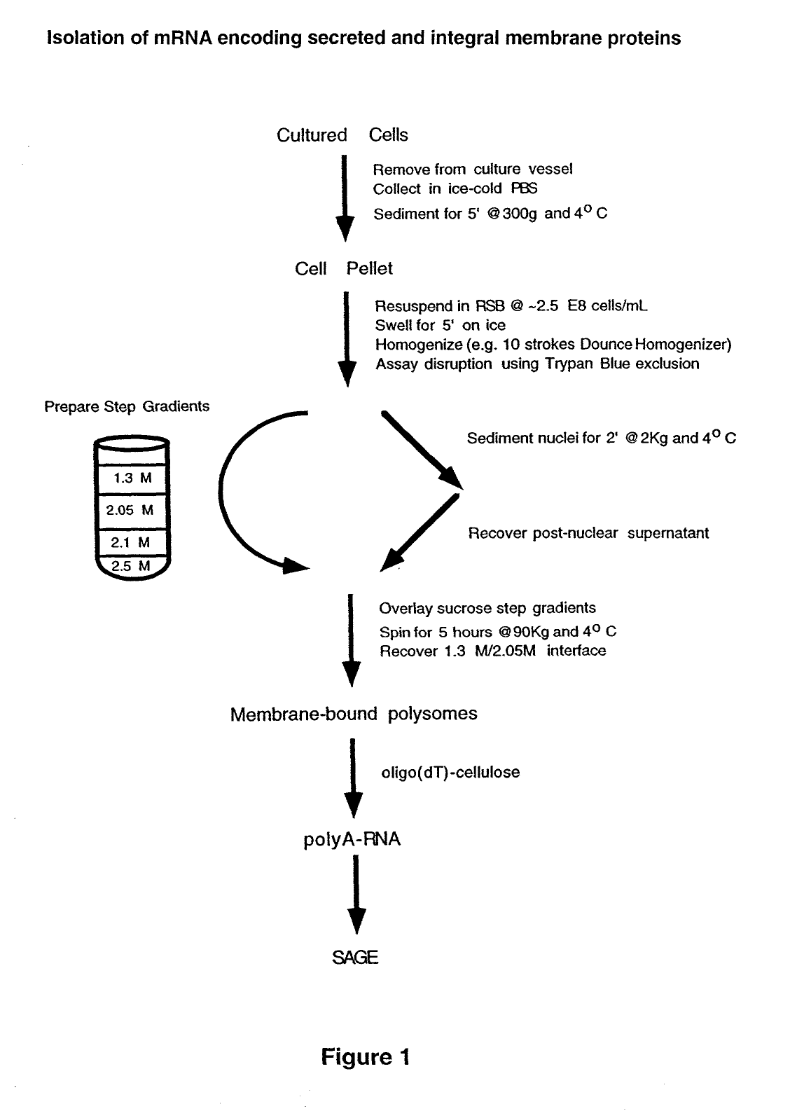

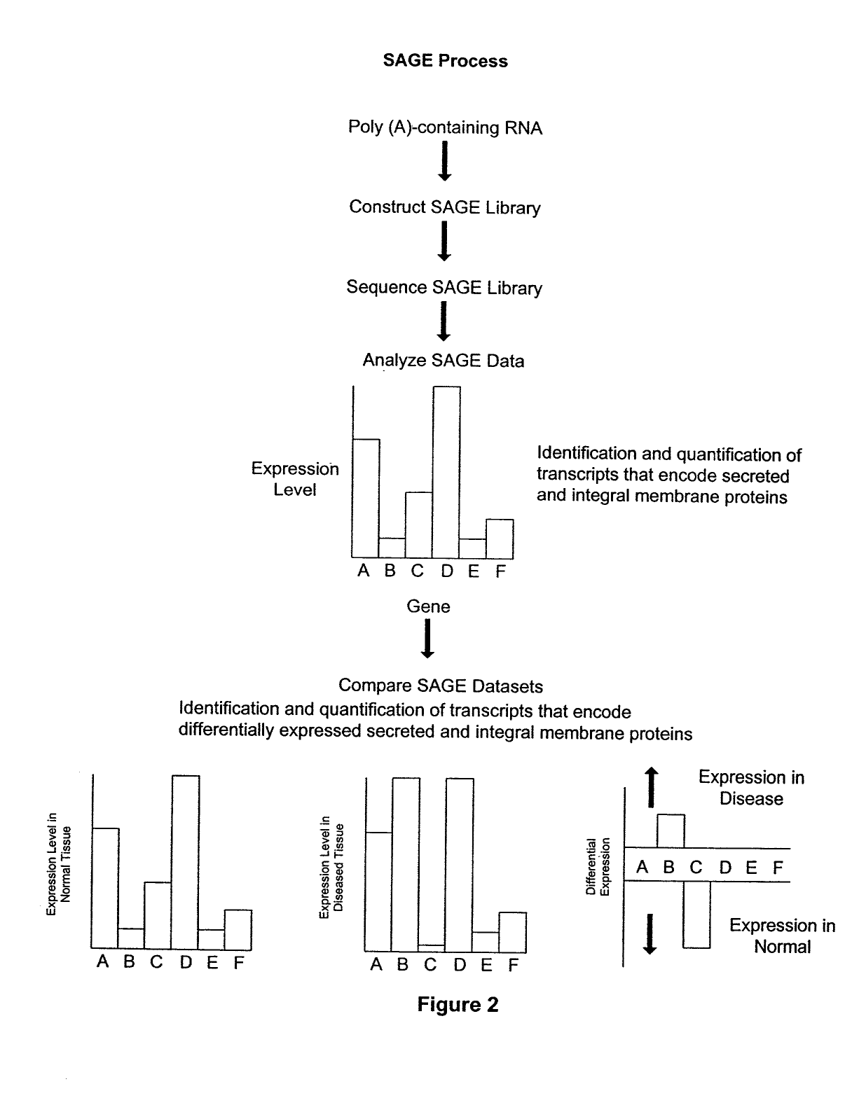

In general, the process requires two phases: (1) enrichment/purification of membrane bound mRNA; and (2) sequence identity. Each of these phases is composed of multiple steps as outlined in FIGS. 1 and 2, respectively. Phase 1 utilizes isopycnic centrifugation to separate cellular or tissue homogenates into 3 fractions, a lighter or microsomal fraction (including membrane bound polysomes) at the 1.3 M/2.05 sucrose interphase, a fraction of intermediate density (2.1 M sucrose) consisting of the free cytoplasmic components, and lastly, the most dense, nuclear fraction at 2.5M. Depending on the cell or tissue type, the membrane bound mRNA may vary from 5-50% of the cellular mRNA. The isopycnic banded material at the 1.3 M/2.5 M sucrose interphase is collected from the top, diluted with aqueous buffer and the RNA further processed by standard procedures to yield polyA-containing RNA.

Phase 2 utilizes poly A-containing RNA from Phase 1 to contruct a library to analyze the polynucleotides, e.g, by SAGE analysis, described in detail below. The SAGE library is sequenced and the resulting sequences analyzed computationally. For this method, the SAGE analysis will accurately define the expression profile of transcripts encoding both integral membrane proteins and secreted proteins. When this analysis is extended to two or more different samples, the results will identify the differences in the expression patter of both integral membrane proteins and secreted proteins.

Additional methods useful in Phase 2 include, but are not limited to differential display and expressed sequence tag methods. The expressed sequence tag (EST) approach is another valuable tool for gene discovery (described in Adams et al. (1991) Science 252:1651), like Northern blotting, RNase protection, and reverse transcriptase-polymerase chain reaction (RT-PCR) analysis (described in Sambrook et al. (1989), supra; Alwine et al. (1977) Proc. Natl. Acad. Sci. USA 74:5350; Zinn et al. (1983) Cell 34:865; and Veres et al. (1987) Science 237:415). A further method utilizes differential display coupled with real time PCT and representational difference analysis (as described in Lisitisyn and Wigler (1995) Meth. Enzymol. 254:291-304). Another approach is the technology known as Serial Analysis of Gene Expression (SAGE), as described in U.S. Pat. No. 5,695,937. By using SAGE, sequence tags (tags being used synonymously with polynucleotides) corresponding to expressed genes can be analyzed.

After isolation of the mRNA that is isolated from the sample, the corresponding cDNA is obtained using methods known to those skilled in the art. In one embodiment, the cDNA is synthesized from mRNA using a biotinylated oligo(dT) primer.

Smaller fragments of cDNA are then created using a restriction endonuclease, preferably one that would be expected to cleave most transcripts at least once. Preferably, a 4-base pair recognition site enzyme is used. More than one restriction endonuclease can also be used, sequentially or in tandem. The cleaved cDNA is isolated by binding to a capture medium for label attached to the primer described above. For example, streptavidin beads are used to isolate the defined 3′ nucleotide sequence polynucleotide when the oligo dT primer for cDNA synthesis is biotinylated. Other capture systems (e.g., biotin/streptavidin, digoxigenin/anti-digoxigenin) can also be employed.

In one aspect, the isolated defined nucleotide sequence polynucleotides are separated into two pools of cDNA. Each pool is ligated using the appropriate linkers. The linkers can be the same or different, although when the linkers have the same sequence, it is not necessary to separate the polynucleotides into pools. The first oligonucleotide linker comprises a first sequence for hybridization of a PCR primer and the second oligonucleotide linker comprises a second sequence for hybridization of a PCR primer. In addition, the linkers further comprise a second restriction endonuclease site. The linkers are designed so that cleavage of the ligation products with the second restriction enzyme results in release of the linker having a defined nucleotide sequence polynucleotide (e.g., 3′ of the restriction endonuclease cleavage site). The defined nucleotide sequence polynucleotide may be from about 6 to 30 base pairs. Preferably, the polynucleotide is about 9 to 11 base pairs. Therefore, a ditag (i.e., the dimer of two sequence tags) is from about 12 to 60 base pairs, and preferably from 18 to 22 base pairs.

Typically, the second restriction endonuclease cleaves at a site distant from or outside of the recognition site. For example, the second restriction endonuclease can be a type IIS restriction enzyme. Type IIS restriction endonucleases cleave at a defined distance up to 20 bp away from their asymmetric recognition sites. See, Szybalski W. (1985) Gene 40:169. Examples of type IIS restriction endonucleases include BsmFI and Fokl. Other similar enzymes are known to those of skill in the art. See, CURRENT PROTOCOLS IN MOLECULAR BIOLOGY, supra.

The pool of defined tags ligated to linkers having the same sequence, or the two pools of defined nucleotide sequence tags ligated to linkers having different nucleotide sequences, are randomly ligated to each other “tail to tail.” The portion of the cDNA polynucleotide furthest from the linker is referred to as the “tail.” This creates the ditag (ligated tag pair) having a first restriction endonuclease site upstream (5′) and a first restriction endonuclease site downstream (3′) of the ditag; a second restriction endonuclease cleavage site upstream and downstream of the ditag, and a linker oligonucleotide containing both a second restriction enzyme recognition site and an amplification primer hybridization site upstream and downstream of the ditag. In other words, the ditag is flanked by the first restriction endonuclease site, the second restriction endonuclease cleavage site and the linkers, respectively.

The ditag can be amplified by utilizing primers that specifically hybridize to one strand of each linker. Preferably, the amplification is performed after the ditags have been ligated together using standard polymerase chain reaction (PCR) methods as described, for example, in U.S. Pat. No. 4,683,195. Alternatively, the ditags can be amplified by cloning in prokaryotic-compatible vectors or by other amplification methods known to those of skill in the art. Those of skill in the art can prepare similar primers for amplification based on the nucleotide sequence of the linkers without undue experimentation.

Cleavage of the amplified PCR product with the first restriction endonuclease allows isolation of ditags that can then be concatenated by ligation. After ligation, it may be desirable to clone the concatemers, although it is not required. Analysis of the ditags or concatemers, whether or not amplification was performed, can be performed by standard sequencing methods. Concatemers generally consist of about 2 to 200 ditags and preferably from about 8 to 20 ditags. While these are preferred concatemers, it will be apparent that the number of ditags that can be concatenated will depend on the length of the individual tags and can be readily determined by those of skill in the art without undue experimentation.

Among the standard procedures for cloning the defined nucleotide sequence tags of the invention is insertion of the tags into vectors such as plasmids or phage. The ditag or concatemers of ditags produced by the method described herein are cloned into recombinant vectors for further analysis, e.g., sequence analysis, plaque/plasmid hybridization using the tags as probes, by methods known to those of skill in the art. Vectors in which the ditags are cloned can be transferred into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

Transformation of a host cell with a vector containing ditag(s) may be carried out by conventional techniques as are well known to those skilled in the art. Where the host is prokaryotic, such as E. coli, competent cells that are capable of DNA uptake can be prepared from cells harvested after exponential growth phase and subsequently treated by the CaCl₂ method using procedures well known in the art. Alternatively, MgCl₂ or RbCl can be used. Electroporation or other commonly used methods in the art can also perform transformation.

Computational Analysis

Although the method is suitably practiced on a single cell type or tissue type, one can also analyze two or more types or the same cell or tissue type before or after therapeutic treatment, for example.

Accordingly, after the polynucleotide information is obtained, it can be analyzed to identify polynucleotides that correspond to genes that are uniquely or differentially expressed between the two or more cell types. It is within the scope of this invention to perform the method described above using previously identified and stored sequence information that define and identify expressed genes. This information can be obtained from private, publicly available and commercially available sequence databases.

For example, after a cell or tissue is selected for having a phenotype which is dependent on the presence of one gene product within a sample cell samples, e.g., cells that secrete a biological factor whose activity can be measured in an in vitro assay, cells that stain with an antibody that recognizes a specific antigen or cells that are lysed by cytotoxic T cells that recognize a specific antigen, the cells are further selected to identify sample cells that exhibit extremes of the chosen phenotype and ideally are matched in all other respects or phenotypic characteristics. For example, cells that are matched, e.g., from the same individual, would minimize having to deal with histocompatability differences.

Ideally one selects two examples of sample cells (say “A” and “B”) that exhibit the chosen phenotype prominently and two examples of samples cells (say “C” and “D”) that do not have the phenotype at all. Using the method of this invention, polynucleotides present in a library form from each cell sample are isolated and their relative expression noted. The individual libraries are sequenced and the information regarding sequence and in some embodiments, relative expression, is stored in any functionally relevant program, e.g., in Compare Report using the SAGE software (available through Dr. Ken Kinzler at Johns Hopkins University, Baltimore, Md.). The Compare Report provides a tabulation of the polynucleotide sequences and their abundance for the samples (say A, B, C and D above) normalized to a defined number of polynucleotides per library (say 25,000). This is then imported into MS-ACCESS either directly or via copying the data into an Excel spreadsheet first and then into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of polynucleotides of specifically defined interest.

The researcher may create groups containing one or more project(s) by combining the counts of specific polynucleotides within a group (e.g., Group Normal=Normal 1+Normal 2; Group Tumor=Primary Tumor1+Tumor Cell Line). Additional characteristic values are also calculated for each tag in the group (e.g., average count, minimum count, and maximum count). The researcher may calculate individual tag count ratios between groups, for example the ratio of the average Group Normal count to the average Group Tumor count for each polynucleotide. The researcher may calculate a statistical measure of the significance of observed differences in tag counts between groups.

To identify the polynucleotides within MS-ACCESS, a query to sort polynucleotide tags based on their abundance in the sample cells is run. The output from the Query report lists specific polynucleotides (by sequence) that fit the sorting criteria and their abundance in the various sample cells.

The sorting is based on the principle that the gene product of interest (and hence the corresponding polynucleotide) is more abundant in the samples that prominently exhibit the chosen phenotype than in samples that do not exhibit the phenotype.

For example, one may query to identify polynucleotides that are present at a level of 10 or more in samples A and B and less than 1 in samples C and D, the results of the search might reveal that 5 different polynucleotides fit the sorting criteria hence there are 5 candidates genes to be tested to determine whether they confer the phenotype when transferred into samples like C and D that do not have the phenotype.

The more stringent the sorting criteria, the more efficient the sorting should be. Thus, if one asked for polynucleotides that are at 5 copies or more in samples A and B and less than 5 copies in samples C and D, a large number of candidates would be generated. However, if one can increase the differential because the samples manifest extremes of the phenotype (say >10 in samples A and B and <1 in samples C and D) this restricts the number of candidates that will be identified.

Prior knowledge of what amount of gene product (hence abundance of polynucleotides) is required to confer the phenotype is not essential as one can arbitrarily select a set of sorting parameters, run the data analysis, and identify and test candidates. If the desired candidate is not found the stringency of the sorting criteria can be reduced (i.e., reduce the differential) and the new candidates that are found can be tested. Iterative cycles of sorting and testing candidates should eventually culminate in the successful recovery of the desired candidate.

TABLE 1

| | Sorting | Number of | Number of Candidates |
|---|---|---|---|
| Cycle | Criteria | Candidates | to Evaluate |
| 1 | ≧10 in samples A and B | 10 | 10 |
| | ≦1 in samples C and D | | |
| | (minimum | | |
| | differential = 10×) | | |
| 2 | ≧5 in samples A and B | 30 | 20* |
| | ≦2 in samples C and D | | |
| | (minimum | | |
| | differential = 2.5×) | | |
| 3 | ≧5 in samples A and B | 80 | 50# |
| | ≦5 in samples C and D | | |
| | (minimum | | |
| | differential = 1×) | | |

*Of the 30 candidates, 10 will have already been evaluated in cycle 1 so only 20 new candidates need to be evaluated
#Of the 80 candidates, 30 will have already been evaluated (10 in cycle 1, 20 in cycle 2) so only 50 need to be evaluated

Knowledge of what amount of gene product (hence abundance of polynucleotide) is required to confer the phenotype will permit the rationale use of stringent sorting criteria and greatly accelerate the search process as the desired gene may be captured within a handful of candidates

Establishing what amount of gene product is required to confer a specific phenotype will be dependent on the specific phenotype in question and the sensitivity of assays that measure that phenotype. For instance, the inventor has found that a frequency of 1/5000 (5 copies of a SAGE tag normalized to a library size of 25,000) correlates with sufficient expression of a tumor antigen within the sample cell to render it sensitive to lysis by an antigen specific T cell while a frequency of 1/25,000 correlates with the cell being weakly sensitive to lysis.

Thus, one could use a sorting criteria of ≧5 in samples cells that are susceptible to lysis and ≦1 in samples that are not susceptible to lysis to home in on a candidate tumor antigen.

Accordingly, one enters the individual polynucleotide sequences from the Query report into the program to determine if there is a match with any known genes or whether they are potentially novel (no match=NM).

Retrieval of cDNAs corresponding to specific sequences from the Query Report are then tested individually in an appropriate biological assay to determine if they confer the phenotype. Of the candidates that correspond to known genes, it is a relatively easy task to obtain complementary DNAs for these candidates and test them individually to determine if they confer the specific phenotype in question when transferred into cells that do not exhibit the phenotype. If none of the known genes confer the phenotype, one can retrieve the cDNAs corresponding to the No Match sequences of the Query Report by PCR cloning and test the novel cDNAs individually for their ability to confer the phenotype. If the assumptions made up to this point are sound (i.e., a single gene product can confer the phenotype; the sorting criteria are not too stringent so as to exclude the desired candidate) then a cDNA corresponding to one of the candidates of the Query Report will be found to confer the phenotype and the search is over. If however none of the candidates are found to confer the phenotype then one may need to reduce the stringency of the sorting parameters to “cast a wider net” and capture more candidates to be tested as above.

In one embodiment, the polynucleotide or gene sequence can also be compared to a sequence database, for example, using a computer method to match a sample sequence with known sequences. Sequence identity can be determined by a sequence comparison using, i.e., sequence alignment programs that are known in the art, such as those described in CURRENT PROTOCOLS IN MOLECULAR BIOLOGY (F. M. Ausubel et al., eds., 1987), Supplement 30, section 7.7.18, Table 7.7.1. Parameters for determining the extent of homology set forth by one or more of the aforementioned alignment programs are well established in the art. They include, but are not limited to p value and percent sequence identity. P value is the probability that the alignment is produced by chance. For a single alignment, the p value can be calculated according to Karlin et al. (1990) Proc. Natl. Acad. Sci. USA 87:2246. For multiple alignments, the p value can be calculated using a heuristic approach such as the one programmed in Blast. The probability of obtaining a statistically significant match between a database and a query sequence increases with the size and diversity of the database and also increases as the query sequence becomes small. Alternatively, the relevance of a statistically significant homology alignment increases as the query sequence becomes longer. To reduce the probability of obtaining random matches with statistically significant, but biologically irrelevant homology, the SAGE tags can be used to obtain larger polynucleotides that are then used for homology analysis.

Polynucleotides Encoding Secreted Proteins

This invention also provides a population of tags or transcripts that are isolated by the method as described herein. This invention further provides larger fragments comprising or corresponding to each individual tag. Using the method identified above, transcripts that transcribe or correspond to secreted proteins can be isolated, identified and characterized. Tags can be computationally compared to sequences such as expressed sequence tags “EST” or known genes. In one aspect, the polynucleotide comprises or corresponds to a polynucleotide that is previously identified but was heretofore not known to have been secreted. In another embodiment, the transcript or gene disclosed herein is “novel,” which means the tag or its respective complement does not comprise a sequence or correspond to a previously identified EST or characterized gene.

The invention also encompasses polynucleotides that differ from that of the polynucleotides described above, but encode substantially the same amino acid sequences. These altered, but phenotypically equivalent polynucleotides are referred to as “functionally equivalent nucleic acids.” As used herein, “functionally equivalent nucleic acids” encompass nucleic acids characterized by slight and non-consequential sequence variations that will function in substantially the same manner to produce the same protein product(s) as the nucleic acids disclosed herein (e.g., by virtue of the degeneracy of the genetic codes), or that have conservative amino acid variations. For example, conservative variations include substitution of a non-polar residue with another non-polar residue, or substitution of a charged residue with a similarly charged residue. These sequence variations include those recognized by artisans in the art as those that do not substantially alter the tertiary structure of the encoded protein. These sequences can be defined by hybridizing to polynucleotides of this invention under conditions of high stringency or by comparing sequence homologies as defined below.

Hybridization can be performed under conditions of different “stringency.” Conditions that vary levels of stringency are well known in the art. See, for example, Sambrook et al., supra. Briefly, relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. In general, a moderate stringency hybridization is typically performed at about 50° C. in 6×SSC, and a high stringency hybridization reaction is generally performed at about 60° C. in 1×SSC.

Alternatively, biologically equivalent polynucleotides can be identified using sequence homology searches. Several embodiments of biologically equivalent polynucleotides are within the scope of this invention, e.g., those characterized by possessing at least 75%, or at least 80%, or at least 90% or at least 95% sequence homology as determined using a sequence alignment program under default parameters correcting for ambiguities in the sequence data, changes in nucleotide sequence that do not alter the amino acid sequence because of degeneracy of the genetic code, conservative amino acid substitutions and corresponding changes in nucleotide sequence, and variations in the lengths of the aligned sequences due to splicing variants or small deletions or insertions between sequences that do not affect function.

A variety of software programs are available in the art. Non-limiting examples of these programs are BLAST family programs, including BLASTN, BLASTP, BLASTX, TBLASTN, and TBLASTX (BLAST is available on the internet at http:/www.ncbi.nlm.nih.gov/BLAST/), FastA, Compare, DotPlot, BestFit, GAP, FrameAlign, ClustalW, and PileUp. These programs can be obtained commercially in a comprehensive package of sequence analysis software such as GCG Inc., Wisconsin Package. Other similar analysis and alignment programs can be purchased from various providers such as DNA Star's MegAlign, or the alignment programs in GeneJockey. Alternatively, sequence analysis and alignment programs can be accessed via the internet at sites such as the CMS Molecular Biology Resource at http://www.sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS. Sequence similarity can be discerned by aligning the tag sequence against a DNA sequence database. Alternatively, the tag sequence can be translated into six reading frames; the predicted peptide sequences of all possible reading frames are then compared to individual sequences stored in a protein database such as s done using the BLASTX program.

Parameters for determining the extent of homology set forth by one or more of the aforementioned alignment programs are well established in the art. They include but are not limited to p value, percent sequence identity and the percent sequence similarity. P value is the probability that the alignment is produced by chance. For a single alignment, the p value can be calculated according to Karlin et al. (1990) Proc. Natl. Acad. Sci. USA 87:2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical. A tag sequence is considered to lack substantial homology with any known sequences when the regions of alignment of comparable length exhibit less than 30% of sequence identity, more preferably less than 20% identity, even more preferably less than 10% identity.

The polynucleotides embodied in the present invention also include larger fragments or the full length coding sequences that encode secreted polypeptides. Based on the novel sequences disclosed herein, fragments or the full length coding sequences of the corresponding novel transcripts or genes can be identified using various cloning methods known to artisans in the art. Five methods are disclosed in the section “Methods of Cloning Novel Transcripts or Genes” which further assist practitioners of ordinary skill to isolate these transcripts, genes or cDNA containing or corresponding to the tag sequences of the invention.

The polynucleotides of the invention can comprise additional sequences, such as additional coding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

Indeed, this invention also provides a promoter sequence derived from cell's genome, wherein the promoter sequence corresponds to the regulatory region of a gene that is identified as described herein. The promoters are identified and characterized by: 1) probing a cDNA library with a probe corresponding to a SAGE tag sequence identified using the method described herein or generating a portion of the desired cDNA by conducting anchored PCR using primers based on the SAGE tag sequence identified by the method described herein. The partial cDNA product obtained in step one above can be used as a probe to clone the extreme 5′ end of the cDNA, and also by using the 5′ end of the cDNA as a probe, cloning from a genomic DNA library the promoter of the gene that encodes the cDNA. Functionally equivalent promoter sequences, as defined above, are further provided by this invention.

The promoters identified above can be operatively linked to a foreign polynucleotide to compel differential transcription of the foreign polynucleotide in the cell from which the promoter was derived. Cells containing these sequences are termed genetically modified cells. In one embodiment, a foreign polynucleotide is any sequence that encodes in whole or in part a polypeptide or protein. It also includes sequences encoding ribozymes and antisense molecules. It further includes regulatory sequences upstream from a gene corresponding to a polynucleotide of this invention.

Also encompassed by this invention are antisense molecules to the polynucleotides described above. Antisense oligonucleotides are useful to inhibit gene expression of the polynucleotides and genes identified by the method of this invention. The construction of antisense molecules or variations thereof is well known in the art. See for example, U.S. Pat. No. 5,958,771. The antisense compounds used in accordance with this invention may be conveniently and routinely made through the well-known technique of solid phase synthesis.

The polynucleotides of the invention can be introduced by any suitable gene delivery method or vector. They also can be expressed in a suitable host cell for generating a cell-based therapy. These methods are described in more detail below.

The polynucleotides and sequences identified above can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates are known which can be employed to provide a means visible to the human eye or spectrophotometrically, to identify specific hybridization with complementary nucleic acid-containing samples.

The polynucleotides and sequences embodied in this invention can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

Compositions containing the polynucleotides and sequences of this invention, in isolated form or contained within a vector or host cell are further provided herein. When these compositions are to be used pharmaceutically, they are combined with a pharmaceutically acceptable carrier.

The polynucleotides and sequences of this invention can be inserted into a suitable vector, and the vector in turn can be introduced into a suitable host cell for replication and/or amplification. Polynucleotides can be introduced into host cells by any means known in the art. Cells are transformed by introducing an exogenous polynucleotide by direct uptake, endocytosis, transfection, f-mating or electroporation. Once introduced, the exogenous polynucleotide can be maintained within the cell as a non-integrated vector (such as a plasmid) or integrated into the host cell genome. Amplified DNA can be isolated from the host cell by standard methods. See, e.g., Sambrook et al. (1989), supra. RNA can also be obtained from transformed host cell, or it can be obtained directly from the DNA by using a DNA-dependent RNA polymerase.

A vector of this invention can contain one or more polynucleotides comprising or corresponding to a sequence identified using the methods described herein. It can also contain polynucleotide sequences encoding other polypeptides that enhance, facilitate, or modulate the desired result, such as fusion components that facilitate protein purification, and sequences that increase immunogenicity of the resultant protein or polypeptide.

Gene delivery vehicles include both viral and non-viral vectors such as naked plasmid DNA or DNA/liposome complexes. Vectors are generally categorized into cloning and expression vectors. Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast and insect expression systems. The polypeptides produced in the various expression systems are also within the scope of the invention and are described above.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller, A. D. et al. (1989) BioTechniques 7:980-990). The methodology of using replication-incompetent retroviruses for retroviral-mediated gene transfer of gene markers is well established (Correll et al. (1989) Proc. Natl. Acad. Sci. USA 86:8912; Bordignon (1989) Proc. Natl. Acad. Sci. USA 86:8912-52; Culver K. (1991) Proc. Natl. Acad. Sci. USA 88:3155; and Rill, D. R. (1991) Blood 79(10):2694. Clinical investigations have shown that there are few or no adverse effects associated with the viral vectors. See, Anderson (1992) Science 256:808-13.

Also embodied in the present invention are host cells transformed with the vectors as described above as well as genetically modified cells as defined above. Both prokaryotic and eukaryotic host cells may be used. Prokaryotic hosts include bacterial cells, for example E. coli and Mycobacteria. Among eukaryotic hosts are yeast, insect, avian, plant and mammalian cells. Host systems are known in the art and need not be described in detail herein. Examples of mammalian host cells include, but are not limited to, COS, HeLa, and CHO cells, and APCs, e.g., dendritic cells.

The host cells of this invention can be used, inter alia, as repositories of polynucleotides differentially expressed in a cell or as vehicles for production of the polynucleotides and the encoded polypeptides.

Also provided by this invention are compositions comprising the host cells and genetically modified cells as described above. In one embodiment, the cells are combined with a carrier such as culture medium for recombinant production of nucleic acid or polypeptide. Additionally, they may be combined with a pharmaceutically acceptable carrier for therapeutic use.

Polypeptides of the Invention

This invention provides a population of proteins or polypeptides expressed from the population of polynucleotides of this invention, which is intended to include wild-type and recombinantly produced polypeptides and proteins from prokaryotic and eukaryotic host cells, as well as muteins, analogs, fusions and fragments thereof. This invention further provides an isolated protein or polypeptide expressed from a polynucleotide of this invention. In some embodiments, the term also includes antibodies and anti-idiotypic antibodies.

It is understood that equivalents or variants of the wild-type polypeptide or protein also are within the scope of this invention. An “equivalent” varies from the wild-type sequence encoded by the polynucleotides of the invention by any combination of additions, deletions, or substitutions while preserving at least one functional property of the fragment relevant to the context in which it is being used. As is apparent to one skilled in the art, the equivalent may also be associated with, or conjugated with, other substances or agents to facilitate, enhance, or modulate its function.

The invention includes modified polypeptides containing conservative or non-conservative substitutions that do not significantly affect their properties, such as the immunogenicity of the peptides or their tertiary structures. Modification of polypeptides is routine practice in the art. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tyrosine. These polypeptides also include glycosylated and nonglycosylated polypeptides, as well as polypeptides with other post-translational modifications, such as, for example, glycosylation with different sugars, acetylation, and phosphorylation.

The polypeptides of the invention can also be conjugated to a chemically functional moiety. Typically, the moiety is a label capable of producing a detectable signal. These conjugated polypeptides are useful, for example, in detection systems such as imaging of tumor tissue. Such labels are known in the art and include, but are not limited to, radioisotopes, enzymes, fluorescent compounds, chemiluminescent compounds, bioluminescent compounds substrate cofactors and inhibitors. See, for examples of patents teaching the use of such labels, U.S. Pat. Nos. 3,817,837; 3,850,752; 3,939,350; 3,996,345; 4,277,437; 4,275,149; and 4,366,241. The moieties can be covalently linked to the polypeptides, recombinantly linked, or conjugated to the polypeptides through a secondary reagent, such as a second antibody, protein A, or a biotin-avidin complex.

The invention also encompasses fusion proteins comprising polypeptides encoded by the polynucleotides disclosed herein and fragments thereof. Such fusion may be between two or more polypeptides of the invention and a related or unrelated polypeptide. Useful fusion partners include sequences that facilitate the intracellular localization of the polypeptide, or enhance immunological reactivity or the coupling of the polypeptide to an immunoassay support or a vaccine carrier. For instance, the polypeptides can be fused with a bioresponse modifier. Examples of bioresponse modifiers include, but are not limited to, fluorescent proteins such as green fluorescent protein (GFP), cytokines or lymphokines such as interleukin-2 (IL-2), interleukin-4 (IL-4), GM-CSF, and α-interferon. Another useful fusion sequence is one that facilitates purification. Examples of such sequences are known in the art, and include those encoding epitopes such as Myc, HA (derived from influenza virus hemagglutinin), His-6, or FLAG. Other fusion sequences that facilitate purification are derived from proteins such as glutathione S-transferase (GST), maltose-binding protein (MBP), or the Fc portion of immunoglobulin. For immunological purposes, tandemly repeated polypeptide segments may be used as antigens, thereby producing highly immunogenic proteins.

The proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

The proteins and polypeptides of this invention are obtainable by a number of processes well known to those of skill in the art, which include purification, chemical synthesis and recombinant methods. Full-length proteins can be purified from a cell or tissue by methods such as immunoprecipitation with antibody, and standard techniques such as gel filtration, ion-exchange, reversed-phase, and affinity chromatography using a fusion protein as shown herein. For such methodology see, for example, Deutscher et al. (1999) GUIDE TO PROTEIN PURIFICATION: METHODS IN ENZYMOLOGY (Vol. 182, Academic Press). Accordingly, this invention also provides the processes for obtaining these proteins and polypeptides as well as the products obtainable and obtained by these processes.

The proteins and polypeptides also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. (1989), supra, using the host cell and vector systems described above.

This invention further provides compositions comprising the polypeptides and proteins of this invention. The polypeptides or proteins can be attached to a solid support for use in purification processes. Additionally, they may be combined with a pharmaceutically acceptable carrier for therapeutic use.

Antibodies

Also provided by this invention are isolated antibodies and a population of antibodies capable of specifically binding to the proteins or polypeptides as described above. The antibodies of the present invention encompass polyclonal antibodies and monoclonal antibodies. They include but are not limited to mouse, rat, and rabbit or human antibodies. This invention also encompasses functionally equivalent antibodies and fragments thereof. As used herein with respect to the exemplified antibodies, the phrase “functional equivalent” means an antibody or fragment thereof, or any molecule having the antigen binding site (or epitope) of the antibody that cross-blocks an exemplified antibody when used in an immunoassay such as immunoblotting or immunoprecipitation.

Antibody fragments include the Fab, Fab′, F(ab′)₂, and Fv regions, or derivatives or combinations thereof. Fab, Fab′, and F(ab′)₂ regions of an immunoglobulin may be generated by enzymatic digestion of the monoclonal antibodies using techniques well known to those skilled in the art. Digesting the monoclonal antibody with papain and contacting the digest with a reducing agent to reductively cleave disulfide bonds may generate fab fragments. Fab′ fragments may be obtained by digesting the antibody with pepsin and reductive cleavage of the fragment so produced with a reducing agent. In the absence of reductive cleavage, enzymatic digestion of the monoclonal with pepsin produces F(ab′)₂ fragments.

It will further be appreciated that encompassed within the definition of antibody fragment is single chain antibody that can be generated as described in U.S. Pat. No. 4,704,692, as well as chimeric antibodies and humanized antibodies (Oi et al. (1986) BioTechniques 4(3):214). Chimeric antibodies are those in which the various domains of the antibodies' heavy and light chains are coded for by DNA from more than one species.

As used herein with regard to the monoclonal antibody, the “hybridoma cell line” is intended to include all derivatives, progeny cells of the parent hybridoma that produce the monoclonal antibodies specific for the polypeptides of the present invention, regardless of generation of karyotypic identity.

Laboratory methods for producing polyclonal antibodies and monoclonal antibodies, as well as deducing their corresponding nucleic acid sequences, are known in the art. See, Harlow and Lane (1988), supra and Sambrook et al. (1989), supra. For production of polyclonal antibodies, an appropriate host animal is selected, typically a mouse or rabbit. The substantially purified antigen, a fragment thereof, alone or fused to another polypeptide, is presented to the immune system of the host by methods appropriate for the host. The antigen is introduced commonly by injection into the host footpads, via intramuscular, intraperitoneal, or intradermal routes. Peptide fragments suitable for raising antibodies may be prepared by chemical synthesis, and are commonly coupled to a carrier molecule (e.g., keyhole limpet hemocyanin) and injected into a host over a period of time suitable for the production of antibodies. Alternatively, the antigen can be generated recombinantly as a fusion protein. Examples of components for these fusion proteins include, but are not limited to myc, HA, FLAG, His-6, glutathione S-transferase, maltose binding protein or the Fc portion of immunoglobulin.

The monoclonal antibodies of this invention refer to antibody compositions having a homogeneous antibody population. It is not intended to be limited as regards to the source of the antibody or the manner in which it is made. Generally, monoclonal antibodies are biologically produced by introducing protein or a fragment thereof into a suitable host, e.g., a mouse. After the appropriate period of time, the spleen of the animal is excised and individual spleen cells fused, typically, to immortalized myeloma cells under appropriate selection conditions. Thereafter the cells are clonally separated and the supernatants of each clone are tested for their production of an appropriate antibody specific for the desired region of the antigen using methods well known in the art.

The isolation of other hybridomas secreting monoclonal antibodies with the specificity of the monoclonal antibodies of the invention can also be accomplished by one of ordinary skill in the art by producing anti-idiotypic antibodies (Herlyn et al. (1986) Science 232: 100). An anti-idiotypic antibody is an antibody that recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody, it is possible to identify other hybridomas expressing monoclonal antibodies of the same epitopic specificity.

It is also possible to use the anti-idiotype technology to produce monoclonal antibodies that mimic an epitope. For example, an anti-idiotypic monoclonal antibody made to a first monoclonal antibody will have a binding domain in the hypervariable region that is the mirror image of the epitope bound by the first monoclonal antibody. Thus, in this instance, the anti-idiotypic monoclonal antibody could be used for immunization for production of these antibodies.

Other suitable techniques of antibody production include, but are not limited to, in vitro exposure of lymphocytes to the antigenic polypeptides or selection of libraries of antibodies in phage or similar vectors. See, Huse et al. (1989) Science 246:1275-1281. Genetically engineered variants of the antibody can be produced by obtaining a polynucleotide encoding the antibody, and applying the general methods of molecular biology to introduce mutations and translate the variant. The above described antibody “derivatives” are further provided herein.

Sera harvested from the immunized animals provide a source of polyclonal antibodies. Detailed procedures for purifying specific antibody activity from a source material are known within the art. Undesired activity cross-reacting with other antigens, if present, can be removed, for example, by running the preparation over adsorbants made of those antigens attached to a solid phase and eluting or releasing the desired antibodies off the antigens. If desired, the specific antibody activity can be further purified by such techniques as protein A chromatography, ammonium sulfate precipitation, ion exchange chromatography, high-performance liquid chromatography and immunoaffinity chromatography on a column of the immunizing polypeptide coupled to a solid support.

The specificity of an antibody refers to the ability of the antibody to distinguish polypeptides comprising the immunizing epitope from other polypeptides. One of ordinary skill in the art can readily determine without undue experimentation, whether an antibody shares the same specificity as a antibody of this invention, by determining whether the antibody being tested prevents an antibody of this invention from binding the polypeptide(s) with which the antibody is normally reactive. If the antibody being tested competes with the antibody of the invention, as shown by a decrease in binding by the antibody of this invention, then it is likely that the two antibodies bind to the same or a closely related epitope. Alternatively, one can pre-incubate the antibody of this invention with the polypeptide(s) with which it is normally reactive, and determine if the antibody being tested is inhibited in its ability to bind the antigen. If the antibody being tested is inhibited, then, in all likelihood, it has the same, or a closely related, epitopic specificity as the antibody of this invention.

The antibodies of the invention can be bound to many different carriers. Thus, this invention also provides compositions containing antibodies and a carrier. Carriers can be active and/or inert. Examples of well-known carriers include, polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies of this invention can also be conjugated to a detectable agent or a hapten. The complex is useful to detect the polypeptide(s) (or polypeptide fragments) to which the antibody specifically binds in a sample, using standard immunochemical techniques such as immunohistochemistry as described by Harlow and Lane (1988), supra. There are many different labels and methods of labeling known to those of ordinary skill in the art. Examples of the types of labels that can be used in the present invention include radioisotopes, enzymes, colloidal metals, fluorescent compounds, bioluminescent compounds, and chemiluminescent compounds. Those of ordinary skill in the art will know of other suitable labels for binding to the antibody, or will be able to ascertain such, using routine experimentation. Furthermore, the binding of these labels to the antibody of the invention can be done using standard techniques common to those of ordinary skill in the art.

Another technique that may also result in greater sensitivity consists of coupling the antibodies to low molecular weight haptens. These haptens can then be specifically detected by means of a second reaction. For example, it is common to use such haptens as biotin, which reacts with avidin, or dinitropherryl, pyridoxal, and fluorescein, which can react with specific anti-hapten antibodies. See Harlow and Lane (1988), supra.

Compositions containing the antibodies, fragments thereof or cell lines which produce the antibodies and a carrier are encompassed by this invention. When these compositions are to be used pharmaceutically, they are combined with a pharmaceutically acceptable carrier.

Uses of Polynucleotides, Polypeptides and Antibodies of the Invention

The polynucleotides identified by the methods described herein can be used to assay expression level of secreted or non-secreted proteins in a cell or tissue sample. In assaying for an alteration in mRNA level, nucleic acid contained in the aforementioned sample is first extracted according to standard methods in the art. For instance, mRNA can be isolated using various lytic enzymes or chemical solutions according to the procedures set forth in Sambrook et al. (1989), supra, or extracted by nucleic-acid-binding resins following the accompanying instructions provided by manufactures. The mRNA contained in the extracted nucleic acid sample is then detected by hybridization (e.g., Northern blot analysis) and/or amplification procedures according to methods widely known in the art or based on the methods exemplified herein.

Nucleotide probes having complementary sequences over stretches greater than 10 nucleotides in length are generally preferred, so as to increase stability and selectivity of the hybrid, and thereby improving the specificity of particular hybrid molecules obtained. More preferably, one can design nucleic acid molecules having gene-complementary stretches of more than 50 nucleotides in length, or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102, or by introducing selected sequences into recombinant vectors for recombinant production. A preferred probe is about 50-75, or more preferably, 50-100, nucleotides in length.

In certain embodiments, it will be advantageous to employ nucleic acid sequences of the present invention in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates are known which can be employed to provide a means visible to the human eye or spectrophotometrically, to identify specific hybridization with complementary nucleic acid-containing samples.

The nucleotide probes of the present invention can also be used as primers and detection of genes or gene transcripts that are differentially expressed in certain body tissues. Additionally, a primer useful for detecting the aforementioned gene or transcript is at least about 80% identical to the homologous region of comparable size of the gene or transcript to be detected contained in the previously identified sequences. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase.

A preferred amplification method is PCR. General procedures for PCR are taught in MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ concentration, ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of the gene or transcript of interest can be verified by demonstrating that the amplified DNA fragment has the predicted size, exhibits the predicated restriction digestion pattern, and/or hybridizes to the correct cloned DNA sequence.

The probes or portions of cognate cDNAs that the tags define as described in this invention also can be attached to a solid support for use in high throughput screening assays using methods known in the art. For example, WO 97/10365 and U.S. Pat. Nos. 5,405,783, 5,412,087 and 5,445,934, disclose the construction of high-density oligonucleotide chips which can contain one or more of the sequences disclosed herein. Based in the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene of interest is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat. Nos. 5,578,832 and 5,631,734. The obtained measurement is directly correlated with gene expression level.

More specifically, the probes and high-density oligonucleotide probe arrays provide an effective means of monitoring expression of a multiplicity of genes. The expression monitoring methods of this invention may be used in a wide variety of circumstances including detection of disease, identification of differential gene expression between two samples, or screening for compositions that upregulate or downregulate the expression of particular genes.

In another preferred embodiment, the methods of this invention are used to monitor expression of the genes that are modulated in response to defined stimuli, such as a drug, by specifically hybridizing the expressed sequences to the probes of this invention.

In one embodiment, the hybridized nucleic acids are detected by detecting one or more labels attached to the sample nucleic acids. The labels may be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

The nucleic acid sample also may be modified prior to hybridization to the high density probe array in order to reduce sample complexity thereby decreasing background signal and improving sensitivity of the measurement using the methods disclosed in WO 97/10365.

Results from the chip assay are typically analyzed using a computer software program. See, for example, EP 0717 113 A2 and WO 95/20681. The hybridization data are read into the program, which calculates the expression level of the targeted gene(s). This figure is compared against existing data sets of gene expression levels for various cell types.

Antibodies that specifically recognize and bind to the protein products of interest are required for conducting the aforementioned protein analyses. These antibodies may be purchased from commercial vendors or generated and screened using methods well known in the art. See, Harlow and Lane (1988), supra, and Sambrook et al. (1989), supra.

There are various methods available in the art for quantifying mRNA or protein level from a cell sample and indeed, any method that can quantify these levels is encompassed by this invention. For example, determination of the mRNA level of the gene may involve, in one aspect, measuring the amount of mRNA in a mRNA sample isolated from the cell by hybridization or quantitative amplification using at least one oligonucleotide probe that is complementary to the mRNA. Determination of the aforementioned protein products requires measuring the amount of immunospecific binding that occurs between an antibody reactive to the product of interest. To detect and quantify the immunospecific binding, or signals generated during hybridization or amplification procedures, digital image analysis systems including but not limited to those that detect radioactivity of the probes or chemiluminescence can be employed.

Screening Assays

The present invention also provides a screen for various agents that modulate the expression of a polynucleotide of this invention. A suitable cell is contacted with an effective amount of a potential agent, and then assayed for a change in the expression level of a polynucleotide identified by the method described herein.

As is apparent to one of skill in the art, suitable cells may be cultured in microtiter plates and several agents may be assayed at the same time by noting genotypic changes and/or phenotypic changes.

When the agent is a composition other than naked DNA or RNA, the agent may be directly added to the cell culture or added to culture medium for addition. As is apparent to those skilled in the art, an “effective” amount must be added which can be empirically determined. When the agent is a polynucleotide, it may be introduced directly into a cell by transfection or electroporation. Alternatively, it may be inserted into the cell using a gene delivery vehicle or other methods as described above.

For the purposes of this invention, an “agent” is intended to include, but not be limited to a biological or chemical compound such as a simple or complex organic or inorganic molecule, a peptide, a protein (e.g. antibody) or a polynucleotide (e.g. anti-sense). A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies.

The assays also can be performed in a subject. When the subject is an animal such as a rat, mouse or simian, the method provides a convenient animal model system that can be used prior to clinical testing of an agent. In this system, a candidate agent is a potential drug if transcript expression is altered, i.e., upregulated (such as restoring tumor suppressor function), downregulated or eliminated as with drug resistant genes or oncogenes, or if symptoms associated or correlated to the presence of cells containing transcript expression are ameliorated, each as compared to untreated, animal having the pathological cells. It also can be useful to have a separate negative control group of cells or animals that are healthy and not treated, which provides a basis for comparison. After administration of the agent to subject, suitable cells or tissue samples are collected and assayed for altered gene expression.

These agents of this invention and the above noted compounds and their derivatives can be combined with a pharmaceutically acceptable carrier for the preparation of medicaments for use in the methods described herein.

The agents of the present invention can be administered to a cell or a subject by various delivery systems known in the art. Non-limiting examples include encapsulation in liposomes, microparticles, microcapsules, expression by recombinant cells, receptor-mediated endocytosis (see, Wu and Wu (1987) J. Biol. Chem. 262:4429-4432), and construction of a therapeutic nucleic acid as part of a retroviral or other vector. Methods of delivery include, but are not limited to transdermally, gene therapy, intra-arterial, intra-muscular, intravenous, intranasal, and oral routes, and include sustained delivery systems. In a specific embodiment, it may be desirable to administer the pharmaceutical compositions of the invention locally to the area in need of treatment; this may be achieved by, for example, and not by way of limitation, local infusion during surgery, by injection, or by means of a catheter or targeted gene delivery of the sequence coding for the therapeutic.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally, transdermally or by inhalation therapy, and may take the form of tablets, lozenges, granules, capsules, pills, ampoules, suppositories or aerosol form. They may also take the form of gene therapy, suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

Functional Analysis

The biological function of the polynucleotides and polypeptides identified by the methods described herein can be demonstrated using various methods commonly employed by artisans of the art. In many cases it is possible to infer the functions of novel genes through comparisons with the functions of known homologs to these genes that are identified using sequence analysis software. In particular, many proteins share variant forms of known functional domains that have been well characterized for their biological activities. These homologies can be identified by basic alignment software tools such as BLAST programs or by more sophisticated approaches such as Profile analysis to identify functional protein motifs with lesser degrees of sequence homology.

Databases and High-throughput Screens

The sequences of polynucleotides of this invention also can be used for comparison to known and unknown sequences using a computer-based method to match a sample sequence with known sequences. Thus, this invention also provides the sequences of the polynucleotides of this invention in a computer database or in computer readable form, including applications utilizing the internet.

A linear search through such a database may be used. Alternatively, the polynucleotide sequence can be converted into a unique numeric representation. The comparison aspects may be implemented in hardware or software, or a combination of both. Preferably, these aspects of the invention are implemented in computer programs executing on a programmable computer comprising a processor, a data storage system (including volatile and non-volatile memory and/or storage elements), at least one input device, and at least one output device. Data input through one or more input devices for temporary or permanent storage in the data storage system includes sequences, and may include previously generated polynucleotides and codes for known and/or unknown sequences. Program code is applied to the input data to perform the functions described above and generate output information. The output information is applied to one or more output devices, in known fashion.

Each such computer program is preferably stored on a storage media or device (e.g., ROM or magnetic diskette) readable by a general or special purpose programmable computer, for configuring and operating the computer when the storage media or device is read by the computer to perform the procedures described herein. The inventive system may also be implemented as a computer-readable storage medium, configured with a computer program, where the storage medium so configured causes a computer to operate in a specific and predefined manner to perform the functions described herein.

A polynucleotide of the invention also can be attached to a solid support for use in high throughput screening assays. For example, WO 97/10365 discloses the construction of high-density oligonucleotide chips. See also, U.S. Pat. Nos. 5,405,783; 5,412,087; and 5,445,934. Using this method, the probes are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See, U.S. Pat. Nos. 5,578,832; and 5,631,734. The obtained measurement is directly correlated with gene expression level.

Results from the chip assay are typically analyzed using a computer software program. See, for example, EP 717,113 A2 and WO 95/20681. The hybridization data is read into the program, which calculates the expression level of the targeted gene(s). This figure is compared against existing data sets of gene expression levels for that cell type.

For example, the database and methods of using the database provides a means to differentiate expression levels and identify novel peptides. Alternatively, the database and methods can be used to distinguish a normal cell (in this case, the reference cell) from a neoplastic cell (i.e., the test cell). It also allows one to differentiate between neoplastic cells biopsied from different regions from a patient or different subjects or gene expression before or after treatment with a potential therapeutic agent. It can be used to analyze drug toxicity and efficacy, as well as to selectively look at protein categories that are expected to be affected by a drug or which may be overexpressed as a result of treatment with a drug, such as the various multi-drug resistant genes. Additional utilities of the database include, but are not limited to analysis of the developmental state of a test cell, the influence of viral or bacterial infection, control of cell cycle, effect of a tumor suppressor gene or lack thereof, polymorphism within the cell type, apoptosis, and the effect of regulatory genes.

Identification of Larger Fragments and Open Reading Frames

Five methods are described below which allow one of skill in the art to isolate a larger polynucleotide, gene or cDNA containing or corresponding to the tags of interest.

RACE-PCR Technique

One method to isolate the gene or cDNA which code for a polypeptide or protein and which corresponds to a transcript of this invention, involves the 5′-RACE-PCR technique. In this technique, the poly-A mRNA that contains the coding sequence of particular interest is first identified by hybridization to a sequence disclosed herein and then reverse transcribed with a 3′-primer comprising the sequence disclosed herein. The newly synthesized cDNA strand is then tagged with an anchor primer of a known sequence, which preferably contains a convenient cloning restriction site attached at the 5′end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc, Clotech) according to the manufacturer's instructions.

Identification of Known Genes or ESTs

In addition, databases exist that reduce the complexity of ESTs by assembling contiguous EST sequences into tentative genes. For example, TIGR has assembled human ESTs into a datable called THC for tentative human consensus sequences. The THC database allows for a more definitive assignment compared to ESTs alone. Software programs are available (TIGR assembler and TIGEM EST assembly machine and contig assembly program (see, Huang, X. (1996) Genomics 33:21-23)) that can assemble ESTs into contiguous sequences from any organism.

Isolation of cDNAs from a Library by Probing with the SAGE Transcript or Tag

Alternatively, mRNA from a sample preparation is used to construct cDNA library in the ZAP Express vector following the procedure described in Velculescu, et al. (1997) Science 270:484. The ZAP Express cDNA synthesis kit (Stratagene) is used accordingly to the manufacturer's protocol. Plates containing 250 to 2000 plaques are hybridized as described in Rupert, et al. (1988) Mol. Cell. Biol. 8:3104 to oligonucleotide probes with the same conditions previously described for standard probes except that the hybridization temperature is reduced to room temperature. Washes are performed in 6×standard-saline-citrate 0.1% SDS for 30 minutes at room temperature. The probes are labeled with ³²P-ATP through use of T4 polynucleotide kinase.

Isolation of Partial cDNA (3′ Fragment) by 3′ Directed PCR Reaction

This procedure is a modification of the protocol described in Polyak, et al. (1997) Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source believed to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 5′-B-TCC GGC GCG CCG TTT T CC CAG TCA CGAT₍₃₀₎-3′ (SEQ ID NO: 1), contains a poly-T stretch at the 3′ end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the streptavidin-coated magnetic beads. Theoretically, any sufficiently-sized DNA region capable of hybridizing to a PCR primer can be used as well as any other 8 base pair recognizing endonuclease.

cDNA constructed utilizing this or similar modified oligo-dT primer is then processed exactly as described in U.S. Pat. No. 5,695,937 up until adapter ligation where only one adapter is ligated to the cDNA pool. After adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

Isolation of cDNA Using GeneTrapper or Modified GeneTrapper Technology

The reagents and manufacturer's instructions for this technology are commercially available from Life Technologies, Inc., Gaithersburg, Md. Briefly, a complex population of single-stranded phagemid DNA containing directional cDNA inserts is enriched for the target sequence by hybridization in solution to a biotinylated oligonucleotide probe complementary to the target sequence. The hybrids are captured on streptavidin-coated paramagnetic beads. A magnet retrieves the paramagnetic beads from the solution, leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded target to double-stranded DNA. Following transformation and plating, typically 20% to 100% of the colonies represent the cDNA clone of interest. To identify the desired cDNA clone, the colonies may be screened by colony hybridization using the ³²P-labeled oligonucleotide as described above for solution hybridization, or alternatively by DNA sequencing and alignment of all sequences obtained from numerous clones to determine a consensus sequence.

The preceding discussion and examples are intended merely to illustrate the art. As it is apparent to one of skill in the art, various modifications can be made to the above without departing from the spirit and scope of this invention.

What is claimed is:

1. A method for identifying a polynucleotide sequence and its expression level, wherein the polynucleotide corresponds to an endoplasmic reticulum-associated polypeptide, comprising the steps of:
(a) obtaining a polynucleotide from a cellular homogenate, wherein the polynucleotide encodes the polypeptide; and
(b) determining the sequence of the nucleic acid and its expression level.

2. The method of claim 1, wherein the nucleic acid of step (a) is isolated by isopycnic centrifugation and isolation of the microsomal fraction.

3. The method of claim 1, wherein step (b) comprises identification of the sequence and its expression level by serial analysis gene expression.

4. The polynucleotide identified by the method of claim 1.

5. A probe comprising at least four contiguous nucleotides of the polynucleotide of claim 4.

6. A coding sequence comprising the polynucleotide of claim 4.

7. A gene comprising the coding sequence of claim 6.

8. A fragment of the coding sequence of claim 6.

9. A fragment of the gene of claim 7.

10. A gene delivery vehicle comprising the polynucleotide of claim 4.

11. A gene delivery vehicle comprising the coding sequence of claim 6.

12. A gene delivery vehicle comprising the gene of claim 8.

13. A host cell comprising the polynucleotide of claim 4.

14. A host cell comprising exogenous added coding sequence of claim 6.

15. A host cell comprising exogenously added gene of claim 8.

16. A composition comprising the polynucleotide of claim 4 and a carrier.

17. A composition comprising host cell comprising the coding sequence of claim 6 and a carrier.

18. A composition comprising the gene of claim 8 and a carrier.

19. An isolated polypeptide encoded by the coding sequence of claim 6.

20. An isolated polypeptide encoded by the gene of claim 8.

21. An antibody that specifically recognizes and binds to the polypeptide of claim 19.

22. An antibody that specifically recognizes and binds to the polypeptide of claim 20.

23. A polynucleotide of claim 4 attached to a chip array.

24. A computer readable medium having encoded thereon the sequence of the polynucleotide of claim 4.

25. A computer readable medium having encoded thereon the coding sequence of the polynucleotide of claim 6.

26. A computer readable medium having encoded thereon the sequence of the gene of claim 8.

27. A method for identifying a polynucleotide coding for a polypeptide associated with the endoplasmic reticulum in a cell, comprising contacting a polynucleotide of claim 4 with nucleic acids isolated from the cell under conditions suitable for amplification and isolation of any nucleic acids complementary to the polynucleotide of claim 4, thereby identifying a polynucleotide coding for a polypeptide associated with the endoplasmic reticulum.

28. A method for identifying a polynucleotide coding for a polypeptide associated with the endoplasmic reticulum in a cell, comprising contacting a polynucleotide of claim 4 with nucleic acids isolated from the cell under conditions suitable for hybridization of complementary sequences and identifying complementary pairs, thereby identifying a polynucleotide coding for a polypeptide associated with the endoplasmic reticulum.

29. A method for identifying a polypeptide associated with the endoplasmic reticulum in a cell, comprising contacting polynucleotides isolated from the cell or tissue containing the cell with the antibody of claim 21 or 22 under conditions such that an antibody-polypeptide complex is formed, and identifying any complex formed, thereby identifying a polypeptide associated with the endoplasmic reticulum.

30. A method for analyzing the effect of an agent on the expression of at least one gene encoding a polypeptide associate with the endoplasmic reticulum in a cell, comprising the steps of:
(a) contacting the agent with the cell and performing the method of claim 1 on the cell after to exposure to the agent; and
(b) comparing the polynucleotides identified in step (a) with polynucleotides identified in a control, thereby analyzing the effect of an agent on the expression of at least one gene encoding a polypeptide associate with the endoplasmic reticulum in a cell.

31. A method for identifying a polynucleotide encoding a non-cytoplasmic protein comprising searching a database for the occurrence of a polynucleotide of claim 4.

32. A method of analyzing an expressed gene in a cell comprising comparing a sequence of a nucleic acid isolated from the cell with a sequence stored on the computer readable medium of claim 25 or 26.
